(19) United States
(12) Patent Application Publication
(10) Pub. No.: US 2025/0072863 A1
(43) Pub. Date: Mar. 6, 2025

(54) Intravascular Ultrasound Catheter

(71) Applicant: Boston Scientific Scimed, Inc., Maple Grove, MN (US)

(72) Inventors: Gordon John Kocur, Lino Lakes, MN (US); Jeffrey Steven Fuller, Brooklyn Park, MN (US); Benjamin Philip Gundale, Plymouth, MN (US); Jeffrey Adam Hammerstrom, New Hope, MN (US)

(73) Assignee: Boston Scientific Scimed, Inc., Maple Grove, MN (US)

(21) Appl. No.: 18/819,001

(22) Filed: Aug. 29, 2024

Related U.S. Application Data
(60) Provisional application No. 63/535,503, filed on Aug. 30, 2023.

(51) Int. Cl. A61B 8/12; A61B 8/00

(52) U.S. Cl. CPC A61B 8/12; A61B 8/445; A61B 8/4466; A61B 8/4483

(57) ABSTRACT

Intravascular imaging catheters and methods for making and using intravascular imaging catheters are disclosed. An example intravascular imaging catheter may include an elongate catheter shaft having a distal end region and a proximal region. The distal end region may include a side port. An imaging core may be disposed within the elongate catheter shaft. The imaging core may include an imaging device configured to be slidable within the elongate catheter shaft and through the side port.

### CROSS-REFERENCE TO RELATED APPLICATIONS

This application claims the benefit of priority under 35 U.S.C. § 119 of U.S. Provisional Application No. 63/535,503, filed Aug. 30, 2023, the entire disclosure of which is hereby incorporated by reference.

### TECHNICAL FIELD

The present disclosure pertains to medical devices, and methods for manufacturing medical devices. More particularly, the present disclosure pertains to imaging devices such as intravascular ultrasound catheters.

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An intravascular imaging catheter is disclosed. The intravascular imaging catheter comprises: an elongate catheter shaft having a distal end region and a proximal region; wherein the distal end region includes a side port; an imaging core disposed within the elongate catheter shaft; and wherein the imaging core includes an imaging device configured to be slidable within the elongate catheter shaft and through the side port.

Alternatively or additionally to any of the embodiments above, the distal end region includes a tip having a guidewire lumen formed therein.

Alternatively or additionally to any of the embodiments above, the distal end region includes a tip and wherein the side port is formed in the tip.

Alternatively or additionally to any of the embodiments above, the imaging device includes an ultrasound transducer.

Alternatively or additionally to any of the embodiments above, the imaging device is configured to image at an angle that is substantially normal to the imaging core.

Alternatively or additionally to any of the embodiments above, the imaging device is configured to image in a distal direction at an angle less than about 90 degrees relative to the imaging core.

Alternatively or additionally to any of the embodiments above, the imaging device includes one or more distally-angled transducers.

Alternatively or additionally to any of the embodiments above, the imaging device is configured to shift between a first orientation and a second orientation.

Alternatively or additionally to any of the embodiments above, the imaging device is configured to image at an angle that is substantially normal to the imaging core when the imaging device is in the first orientation.

Alternatively or additionally to any of the embodiments above, the imaging device is configured to image in a distal direction at an angle less than about 90 degrees relative to the imaging core when the imaging device is in the second orientation.

Alternatively or additionally to any of the embodiments above, the imaging device is configured to image in a distal direction at a variable angle.

Alternatively or additionally to any of the embodiments above, the imaging device is mounted upon an inflatable member.

Alternatively or additionally to any of the embodiments above, the imaging device includes a plurality of transducers.

Alternatively or additionally to any of the embodiments above, the inflatable member has a guidewire lumen formed therein.

Alternatively or additionally to any of the embodiments above, the elongate catheter shaft includes a torque-transmitting reinforcing member.

An intravascular imaging catheter is disclosed. The intravascular imaging catheter comprises: an elongate catheter shaft having a distal end region and a proximal region; wherein the proximal region includes a torque-transmitting reinforcing member; wherein the distal end region includes a side port; an imaging core disposed within the elongate catheter shaft; and wherein the imaging core includes one or more ultrasound transducers configured to be slidable within the elongate catheter shaft and through the side port.

Alternatively or additionally to any of the embodiments above, the one or more ultrasound transducers include one or more distally-angled transducers.

Alternatively or additionally to any of the embodiments above, the one or more ultrasound transducers are configured to shift between a first orientation and a second orientation.

Alternatively or additionally to any of the embodiments above, the one or more ultrasound transducers are configured to image at a first angle that is substantially normal to the imaging core when the one or more ultrasound transducers are in the first orientation and wherein the one or more ultrasound transducers are configured to image in a distal direction at a second angle less than about 90 degrees relative to the imaging core when the one or more ultrasound transducers are in the second orientation.

A method for imaging a vascular region is disclosed. The method comprises: advancing an intravascular imaging catheter through a blood vessel to a position adjacent to an area of interest; wherein the intravascular imaging catheter comprises: an elongate catheter shaft having a distal end region and a proximal region, wherein the distal end region includes a side port, an imaging core disposed within the elongate catheter shaft, and wherein the imaging core includes an imaging device configured to be slidable within the elongate catheter shaft and through the side port; advancing the imaging core so that the imaging device advances through the side port; and imaging the blood vessel using the imaging device.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of a portion of an example medical device.

FIG. 2 is a side view of a portion of an example medical device.

FIGS. 3-4 are side views of a portion of an example medical device.

FIGS. 5-7 are side views of a portion of an example medical device.

FIG. 8 is a side view of a portion of an example medical device.

FIG. 9 is a side view of a portion of an example medical device.

FIG. 10 is a side view of a portion of an example medical device.

FIG. 11 is a side view of a portion of an example medical device.

FIG. 12 is a side view of a portion of an example medical device.

FIG. 13 is a side view of a portion of an example medical device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

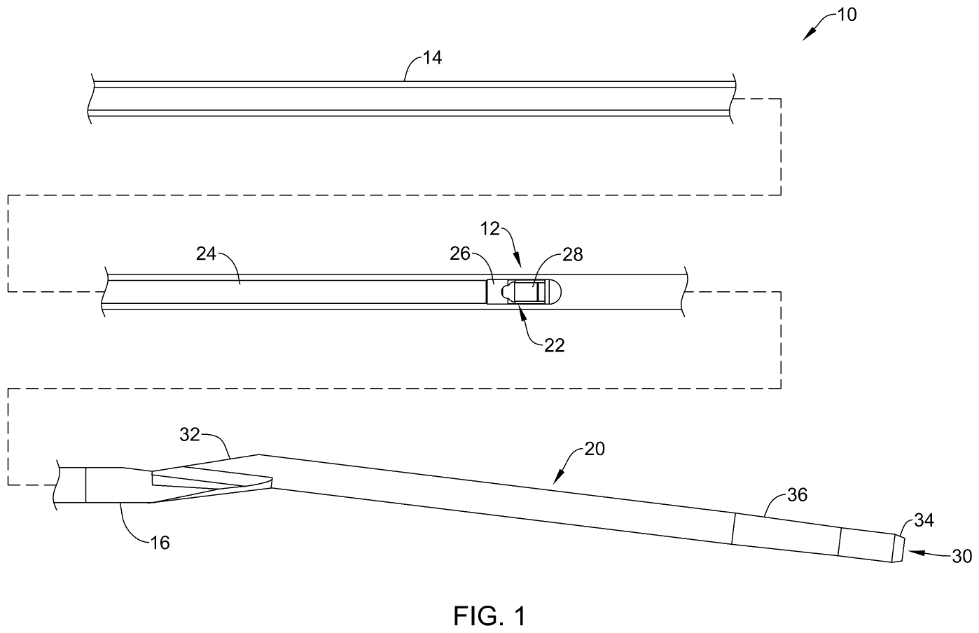

FIG. 1 is a side view of a portion of an example medical device 10. In at least some instances, the medical device 10 takes the form of an imaging medical device. For example, the medical device 10 may be an intravascular ultrasound (IVUS) device that may be used to image a blood vessel. In some of these and in other instances the medical device may be an optical coherence tomography (OCT) imaging device, a near-infrared spectroscopy (NIRS) imaging device, near-infrared fluorescence (NIRF) imaging device, a photoacoustic imaging device, a fluorescence-lifetime imaging device, combinations thereof (including combinations that include IVUS), and/or the like. In addition to be used for intravascular imaging, the medical device 10 may also be used for pulmonary procedures/imaging. The structure/form of the medical device 10 can vary. In some instances, the medical device 10 may include an elongate catheter shaft 12 having a proximal end region 14 and a distal end region 16. A tip member 20 may be coupled to or otherwise disposed adjacent to the distal end region 16. The tip member 20 may include a guidewire lumen 30 having a guidewire exit port 32, an atraumatic distal end 34, one or more radiopaque markers 36, and/or other features. In some embodiments, the tip member 20 may extend at a non-parallel angle to the proximal end region 14 of the elongate catheter shaft 12.

An imaging assembly 22 (e.g., which may sometime be referred to as an imaging core) may be disposed within a lumen of the elongate catheter shaft 12. In general, the imaging core 22 may be used to capture/generate images of a blood vessel. In some instances, the medical device may include devices and/or features similar to those disclosed in U.S. Patent Application Pub. No. US 2012/0059241 and U.S. Patent Application Pub. No. US 2017/0164925, the entire disclosures of which are herein incorporated by reference. In at least some instances, the medical device 10 may resemble and/or include features that resemble the OPTICROSS™ Imaging Catheter, commercially available from BOSTON SCIENTIFIC, Marlborough, MA.

The imaging core 22 may include a drive shaft or cable 24, a housing 26, and an imaging member or transducer 28 coupled to the drive shaft 24 and/or housing 26. In at least some instances, the transducer 28 includes an ultrasound transducer. Other transducers are also contemplated. The transducer 28 may be rotatable and/or axially translatable relative to the elongate catheter shaft 12. For example, the drive shaft 24 may be rotated and/or translated in order to rotate and/or translate the transducer 28 (and the housing 26).

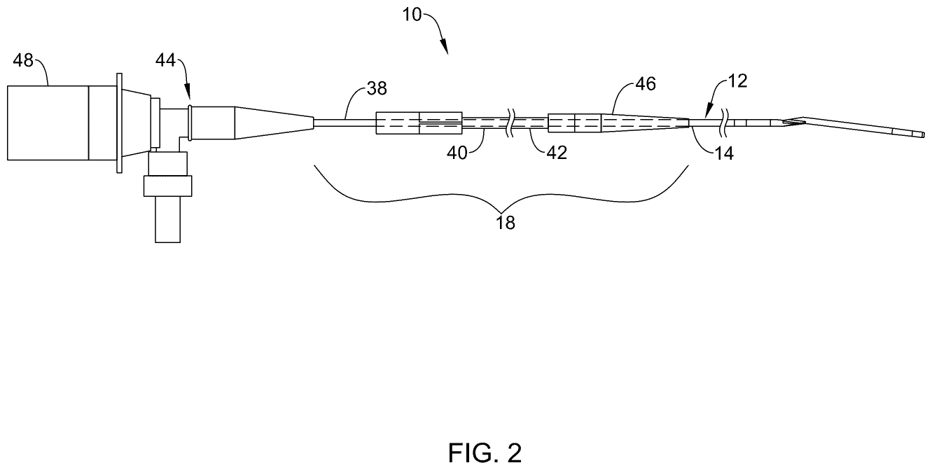

In some instances, the proximal end region 14 of the elongate catheter shaft 12 may be coupled to a telescoping assembly 18 as shown in FIG. 2. In general, the telescoping assembly 18 may be configured to allow the medical device operator to move the drive shaft 24 including the imaging core 22 proximally and distally within the catheter (e.g., relative to the elongate catheter shaft 12), without having to move the entire catheter within the patient. This allows the catheter operator to easily change the location of the imaging core 22 within the patient. For example, the telescoping assembly 18 may be actuated to change the location of the imaging core 22 within the elongate catheter shaft 12.

The proximal end region 14 of the elongate catheter shaft 12 may be coupled to the telescoping assembly 18. For example, the proximal end region 14 of the elongate catheter shaft 12 may be coupled to a distal hub 46 of the telescoping assembly 18. A proximal hub 44 may be coupled to the telescoping assembly 18 (e.g., at the proximal end of the telescoping assembly 18). The drive shaft 24 (see FIG. 1) may extend through the telescoping assembly 18 and be coupled to and/or otherwise secured to the proximal hub 44. The proximal hub 44 may include a connector assembly 48. In general, the connector assembly 48 may allow the medical device 10 (e.g., the elongate catheter shaft 12) to a control unit (e.g., a motor drive unit and/or the like) as described in more detail herein.

The telescoping assembly 18 may include a first sheath 38 and a second sheath 40. In some instances, the first sheath 38 may be understood to be an inner telescoping tube 38 and the second sheath 40 may be understood to be an outer telescoping tube 40. Generally, the outer telescoping tube 40 may be disposed over the inner telescoping tube 38. The inner telescoping tube 38 may be coupled to or otherwise secured to the proximal hub 44. The outer telescoping tube 40 may be coupled or otherwise secured to the distal hub 46. The inner telescoping tube 38 may be axially and/or rotatably moveable relative to the outer telescoping tube 40. Because the drive shaft 24 may be secured to the proximal hub 44 and/or the inner telescoping tube 38 and because the elongate catheter shaft 12 may be secured to the distal hub 46, movement of the proximal hub 44 relative to the distal hub 46 results in movement of the inner telescoping tube 38 and the drive shaft 24 relative to the distal hub 46 and/or the elongate catheter shaft 12.

During an intravascular intervention, the medical device 10 may encounter a number of anatomical structures such as vessel bifurcations and/or occlusions (e.g., chronic total occlusions). If the imaging device 10 is oriented at an angle that generally points to the side or periphery of the medical device 10, it may be challenging to image such structures (e.g., which may be oriented distally and/or otherwise “forward” of the imaging device 10). It may be desirable for an imaging device to be able to image in a substantially forward direction. Disclosed herein are medical devices that include imaging devices that are designed to image in a substantially forward direction. Some additional details of such medical devices are also disclosed herein.

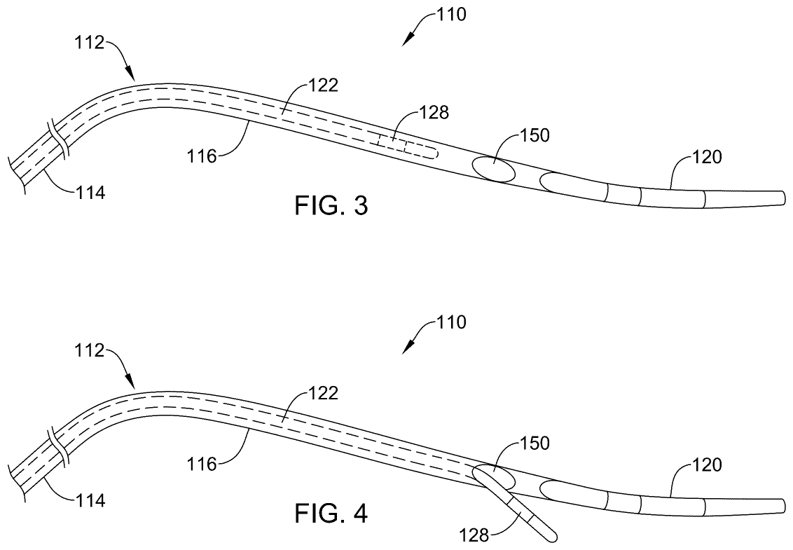

FIGS. 3-4 illustrate a portion of another example medical device 110 that may be similar in form and function to other medical device disclosed herein. In some instances, the medical device 110 may be an intravascular imaging device 110. The intravascular imaging device 110 may include an elongate catheter shaft or imaging sheath 112. The elongate catheter shaft 112 may include a proximal region 114 and a distal end region 116. The elongate catheter shaft 112 may also include a distal tip region 120.

An imaging core 122 may be disposed (e.g., slidably disposed) within the elongate catheter shaft 112. The imaging core 122 may include an imaging device 128. In at least some instances, the imaging device 128 may include an ultrasound imaging device (e.g., an ultrasound transducer). Other imaging devices are contemplated. For example, the imaging device 128 may include an OCT device, a NIRS device, a NIRF device, and/or combinations thereof (e.g., including combinations that may include an IVUS imaging device).

A side port 150 may be formed or defined in the elongate catheter shaft 112. In this example, the side port 150 may be formed in the distal end region 116. The side port 150 may be formed in a side wall of the elongate catheter shaft 112. In some instances, the imaging core 122 may be configured to shift between a delivery position (e.g., as shown in FIG. 3) where the imaging device 128 is disposed within the distal end region 116 and an imaging position (e.g., as shown in FIG. 4) where the imaging core 122 is extended through the side port 150 so that the imaging device 128 is oriented in a distal or forward direction.

It can be appreciated that because the side port 150 may be formed in a side wall of the elongate catheter shaft 112, as the imaging core 122 is advanced through the side port 150, the imaging core 122 will tend to arc, bend, and/or curve. Because of this, rather than the imaging device 128 being oriented radially or at an angle that is substantially normal to the longitudinal axis of the imaging core 122, the imaging device 128 may be disposed at an angle that has at least a component that can be considered to be distally-oriented.

In some instances, the imaging core 122 can be rotated in order to orient the imaging device 128 in a desired direction during an imaging procedure. This may include rotating the imaging core 122 to a desired orientation in order to be view/image the target region of interest. Rotation can occur while the imaging device 128 is still disposed within the elongate catheter shaft 112 and/or after the imaging device 128 is advanced through the side port 150. In some of these and in other instances, the elongate catheter shaft 112 can rotate in order to orient the imaging device 128 in the desired direction. The elongate catheter shaft 112 may include a torque-transmitting reinforcing member (e.g., a braid, a cut tube, a cut hypotube, and/or the like) to facilitate rotation.

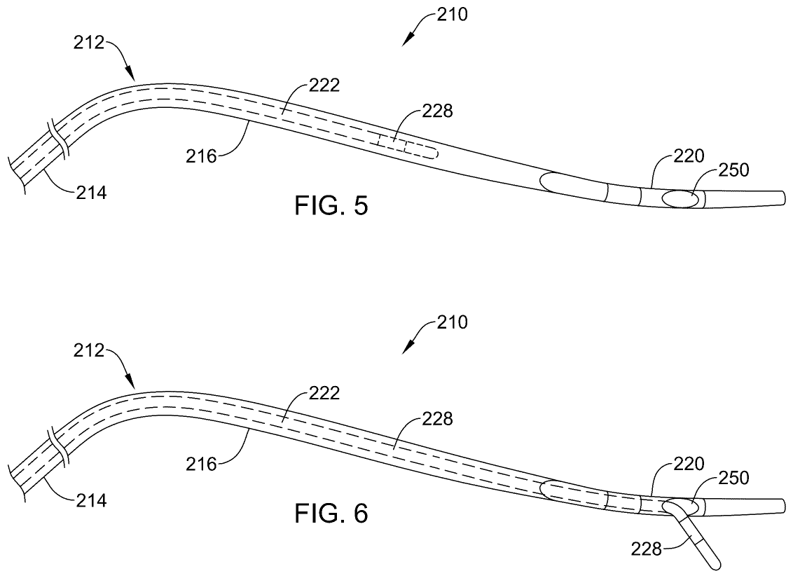

FIGS. 5-7 illustrate a portion of another example medical device 210 that may be similar in form and function to other medical device disclosed herein. In some instances, the medical device 210 may be an intravascular imaging device 210. The intravascular imaging device 110 may include an elongate catheter shaft or imaging sheath 212. The elongate catheter shaft 212 may include a proximal region 214 and a distal end region 216. The elongate catheter shaft 212 may also include a distal tip region 220.

An imaging core 222 may be disposed (e.g., slidably disposed) within the elongate catheter shaft 212. The imaging core 222 may include an imaging device 228. In at least some instances, the imaging device 228 may include an ultrasound imaging device (e.g., an ultrasound transducer). Other imaging devices are contemplated. For example, the imaging device 228 may include an OCT device, a NIRS device, a NIRF device, and/or combinations thereof (e.g., including combinations that may include an IVUS imaging device).

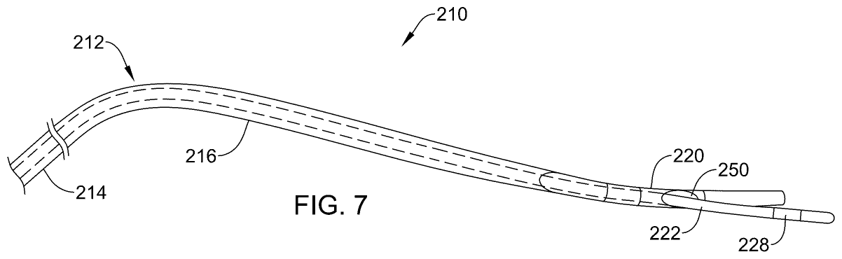

A side port 250 may be formed or defined in the elongate catheter shaft 212. In this example, the side port 250 may be formed in the distal tip region 220. In some instances, the imaging core 222 may be configured to shift between a delivery position (e.g., as shown in FIG. 5) where the imaging device 228 is disposed within the distal end region 216 and an imaging position (e.g., as shown in FIG. 6) where the imaging core 222 is extended through the side port 250 so that the imaging device 128 is oriented in a distal or forward direction. In some instances, the imaging core 222 can be further extended as shown in FIG. 7. This may include advancing the imaging core 222 so that at least a portion of the imaging core 22 extends distally beyond a distal end of the elongate catheter shaft 212. In some instances, the imaging device 228 may be positioned just proximal of the distal end of the elongate catheter shaft 212, at a position that is substantially aligned with the distal end of the elongate catheter shaft 212, or at a position that extends distally beyond a distal end of the elongate catheter shaft 212.

In some instances, the imaging core 222 can be rotated in order to orient the imaging device 228 in a desired direction during an imaging procedure. This may include rotating the imaging core 222 to a desired orientation in order to be view/image the target region of interest. Rotation can occur while the imaging device 228 is still disposed within the elongate catheter shaft 212 and/or after the imaging device 228 is advanced through the side port 250. In some of these and in other instances, the elongate catheter shaft 212 can rotate in order to orient the imaging device 228 in the desired direction. The elongate catheter shaft 212 may include a torque-transmitting reinforcing member (e.g., a braid, a cut tube, a cut hypotube, and/or the like) to facilitate rotation.

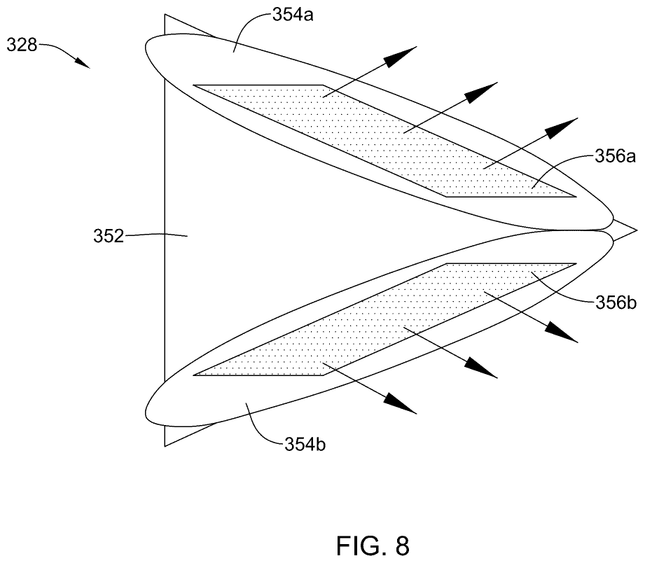

FIG. 8 illustrates a portion of an imaging device 328 that can be used with any of the devices and/or imaging cores disclosed herein. In this example, the imaging device 328 may include a housing 352 and one or more transducer regions 354a, 354b. Transducers 356a, 356b may be disposed along the transducer regions 354a, 354b. For the purposes of this disclosure, a laterally oriented transducer may be understood to image in a direction that is substantially normal to the longitudinal axis of the imaging core. As can be seen, rather than having one or more transducers that point laterally, the transducers 356a, 356b are distally-oriented. In other words, the transducers 356a, 356b may be understood to be configured to image in a distal direction at an angle less than about 90 degrees relative to the imaging core. In FIG. 8, arrows are shown to denote the direction that the transducers 356a, 356b can image. This orientation can be understood to be in a distal direction at an angle less than about 90 degrees relative to the imaging core.

The imaging device 328 can be secured to a drive cable (e.g., similar to the drive cable 24), be part of an imaging core, and/or be slidably disposed within an elongate catheter shaft. This may include structures similar to those disclosed herein. In some instances, the drive cable (and/or the imaging core, in general) can be used to rotate the imaging device 328 during an imaging procedure. This may include rotating the imaging device 328 to a desired orientation in order to be view/image the target region of interest. Rotation can occur while the imaging device 328 is disposed within the elongate catheter shaft. In instance where the elongate catheter shaft includes a side port, rotation can occur after the imaging device 328 is advanced through the side port. In some of these and in other instances, the elongate catheter shaft can rotate in order to orient the imaging device 328 in the desired direction. The elongate catheter shaft may include a torque-transmitting reinforcing member (e.g., a braid, a cut tube, a cut hypotube, and/or the like) to facilitate rotation.

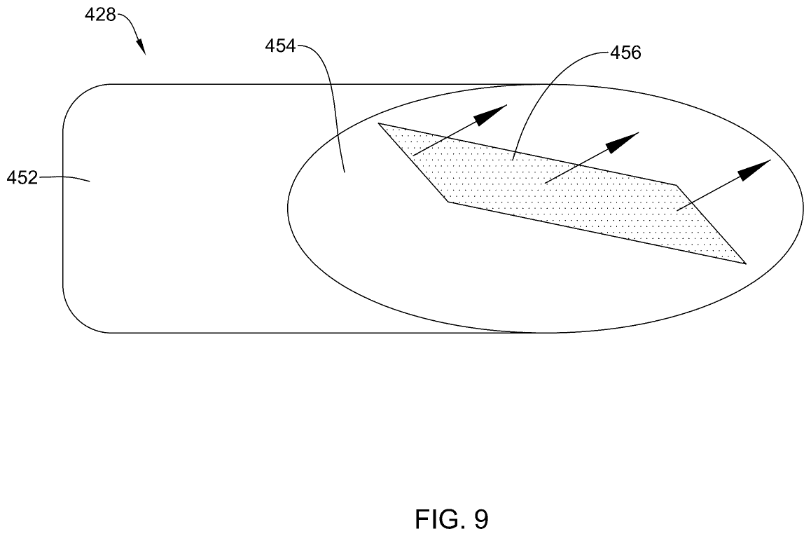

FIG. 9 illustrates a portion of an imaging device 428 that can be used with any of the devices and/or imaging cores disclosed herein. In this example, the imaging device 428 may include a housing 452 and a transducer region 454. A transducer 456 may be disposed along the transducer region 454. As can be seen, rather than having one or more transducers that point laterally, the transducers 456 is distally-oriented. In other words, the transducer 456 may be understood to be configured to image in a distal direction at an angle less than about 90 degrees relative to the imaging core. In FIG. 8, arrows are shown to denote the direction that the transducer 456 can image. This orientation can be understood to be in a distal direction at an angle less than about 90 degrees relative to the imaging core.

The imaging device 428 can be secured to a drive cable (e.g., similar to the drive cable 24), be part of an imaging core, and/or be slidably disposed within an elongate catheter shaft. This may include structures similar to those disclosed herein. In some instances, the drive cable (and/or the imaging core, in general) can be used to rotate the imaging device 428 during an imaging procedure. This may include rotating the imaging device 428 to a desired orientation in order to be view/image the target region of interest. Rotation can occur while the imaging device 428 is disposed within the elongate catheter shaft. In instance where the elongate catheter shaft includes a side port, rotation can occur after the imaging device 428 is advanced through the side port. In some of these and in other instances, the elongate catheter shaft can rotate in order to orient the imaging device 428 in the desired direction. The elongate catheter shaft may include a torque-transmitting reinforcing member (e.g., a braid, a cut tube, a cut hypotube, and/or the like) to facilitate rotation.

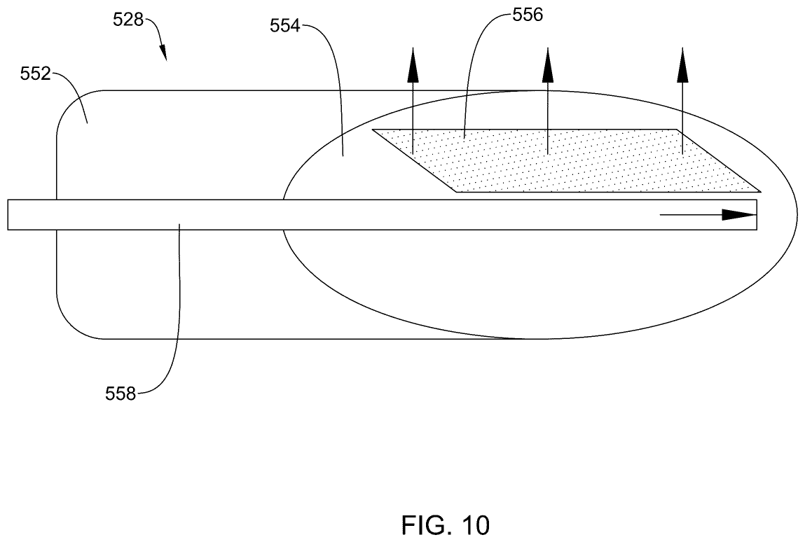

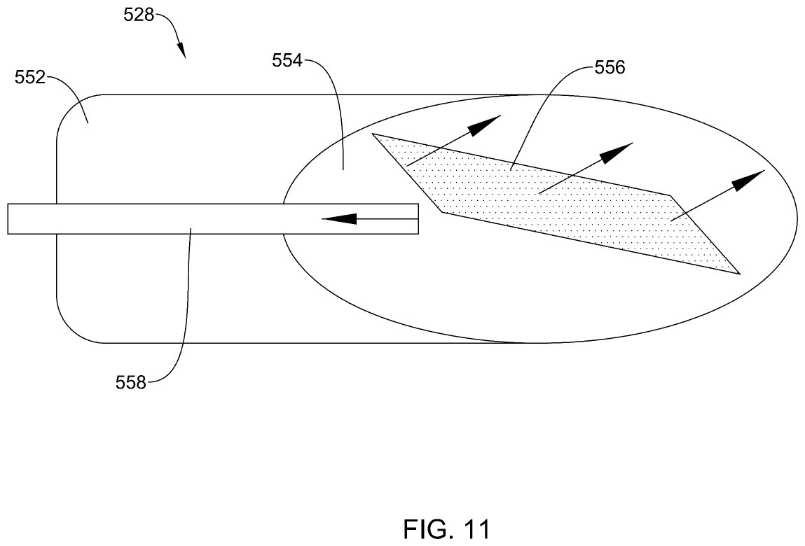

FIGS. 10-11 illustrate a portion of an imaging device 528 that can be used with any of the devices and/or imaging cores disclosed herein. In this example, the imaging device 528 may include a housing 552 and a transducer region 554. A transducer 556 may be disposed along the transducer region 554. In this example, the transducer 556 may be shiftable between a first orientation (e.g., as shown in FIG. 10) and a second orientation (e.g., as shown in FIG. 11). An actuator 558 may be disposed along the imaging device 528. The actuator 558 may take the form of a wire or shaft that can tilt or otherwise shift the transducer 556 between a first orientation (e.g., as shown in FIG. 10) and a second orientation (e.g., as shown in FIG. 11). For example, when the actuator 558 is disposed at a distal position (e.g., as shown in FIG. 10), the transducer 556 may image at an angle that is substantially normal to the longitudinal axis of the imaging device 528 (and/or an imaging core). When the actuator 558 is shifted (e.g., proximally; as shown in FIG. 11), the transducer 556 may be understood to be configured to image in a distal direction at an angle less than about 90 degrees relative to the imaging core.

The imaging device 528 can be secured to a drive cable (e.g., similar to the drive cable 24), be part of an imaging core, and/or be slidably disposed within an elongate catheter shaft. This may include structures similar to those disclosed herein. In some instances, the drive cable (and/or the imaging core, in general) can be used to rotate the imaging device 528 during an imaging procedure. This may include rotating the imaging device 528 to a desired orientation in order to be view/image the target region of interest. Rotation can occur while the imaging device 528 is disposed within the elongate catheter shaft. In instance where the elongate catheter shaft includes a side port, rotation can occur after the imaging device 528 is advanced through the side port. In some of these and in other instances, the elongate catheter shaft can rotate in order to orient the imaging device 528 in the desired direction. The elongate catheter shaft may include a torque-transmitting reinforcing member (e.g., a braid, a cut tube, a cut hypotube, and/or the like) to facilitate rotation.

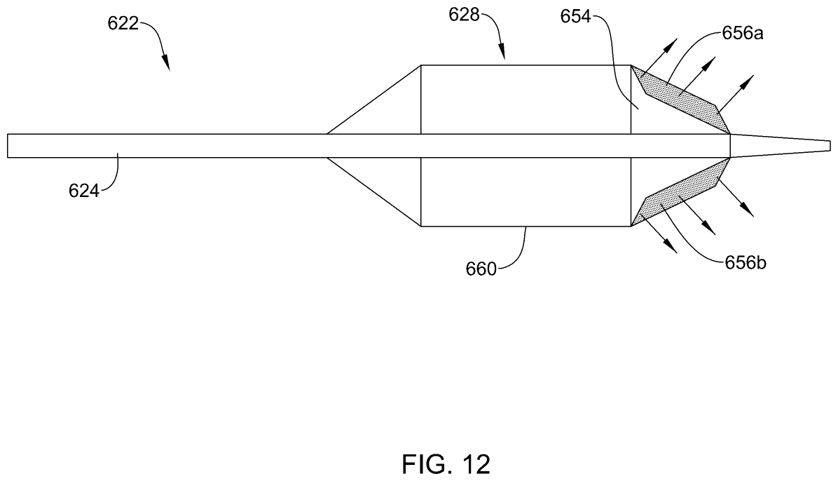

FIG. 12 illustrates a portion of an imaging core 622 that may be similar in form and function to other imaging cores disclosed herein. The imaging core 622 may include a drive shaft 624 and an imaging device 628. In this example, the imaging device may include an expandable member 660 such as a balloon. The balloon 660 may include a transducer region 654. The transducer region 654 may be distal cone of the balloon 660. Transducers 656a, 656b may be disposed along the transducer region 654.

It can be appreciated that disposing the transducers 656a, 656b along distal cone region (e.g., the transducer region 654) of the balloon 660 may allow the angle/orientation of the transducers 656a, 656b to vary. For example, varying the amount of inflation of the balloon 660 will shift the orientation of the transducer region 654. For example, when the balloon 660 is less inflated, the transducer region 654 may be disposed closer to drive shaft 624. This may cause the transducers 656a, 656b to image at a higher angle (an angle that gets closer to 90 degrees as the balloon 660 inflation level drops). As the balloon 660 inflates more, the transducer region 654 may shift further away from the drive shaft 624. This may cause the transducers 656a, 656b to image at a shallower angle (an angle that gets closer to 0 degrees as the balloon 660 inflation level increases).

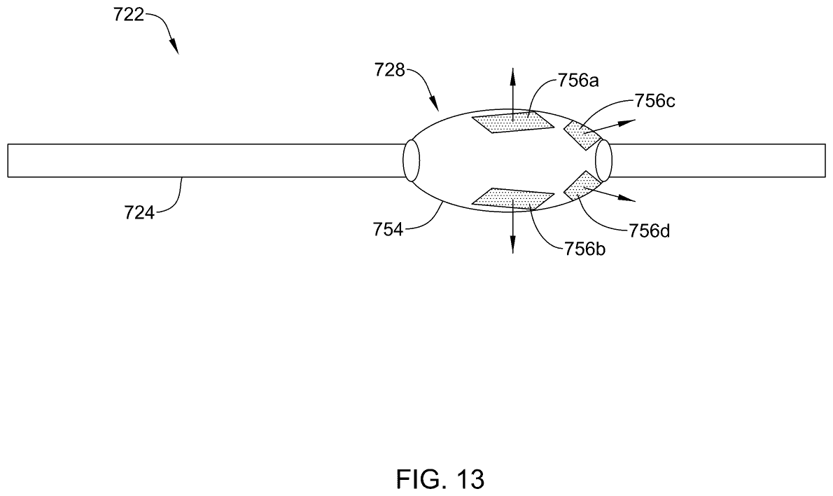

FIG. 13 illustrates a portion of an imaging core 722 that may be similar in form and function to other imaging cores disclosed herein. The imaging core 722 may include a drive shaft 724 and an imaging device 728. In this example, the imaging device 728 may include an expandable member such as a balloon having a transducer region 754. Transducers 756a, 756b, 756c, 756d may be disposed along the transducer region 754. The transducers 756a, 756b may image at an angle that is substantially normal to the longitudinal axis of the imaging device 728 (and/or an imaging core and/or the drive shaft 724). The transducers 756c, 756d may be understood to be configured to image in a distal direction at an angle less than about 90 degrees relative to the imaging core (e.g., and/or the drive shaft 724). Just like the transducers 656a, 656b, the transducers 756c, 756d may vary in angle/orientation, depending on the inflation level of the balloon.

The materials that can be used for the various components of the medical device 10 (and/or other medical devices disclosed herein) may include those commonly associated with medical devices. For simplicity purposes, the following discussion makes reference to the elongate catheter shaft 12 and other components of the medical device 10. However, this is not intended to limit the devices and methods described herein, as the discussion may be applied to other shafts and/or catheters disclosed herein.

The elongate catheter shaft 12 and/or other components of the medical device 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly (alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the medical device 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the medical device 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device 10 to achieve the same result.

In some embodiments, a degree of Magnetic Resonance Imaging (MRI) compatibility is imparted into the medical device 10. For example, the medical device 10, or portions thereof, may be made of a material that does not substantially distort the image and create substantial artifacts (e.g., gaps in the image). Certain ferromagnetic materials, for example, may not be suitable because they may create artifacts in an MRI image. The medical device 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-NR and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An intravascular imaging catheter, comprising:
an elongate catheter shaft having a distal end region and a proximal region;
wherein the distal end region includes a side port;
an imaging core disposed within the elongate catheter shaft; and
wherein the imaging core includes an imaging device configured to be slidable within the elongate catheter shaft and through the side port.

2. The intravascular imaging catheter of claim 1, wherein the distal end region includes a tip having a guidewire lumen formed therein.

3. The intravascular imaging catheter of claim 1, wherein the distal end region includes a tip and wherein the side port is formed in the tip.

4. The intravascular imaging catheter of claim 1, wherein the imaging device includes an ultrasound transducer.

5. The intravascular imaging catheter of claim 1, wherein the imaging device is configured to image at an angle that is substantially normal to the imaging core.

6. The intravascular imaging catheter of claim 1, wherein the imaging device is configured to image in a distal direction at an angle less than about 90 degrees relative to the imaging core.

7. The intravascular imaging catheter of claim 1, wherein the imaging device includes one or more distally-angled transducers.

8. The intravascular imaging catheter of claim 1, wherein the imaging device is configured to shift between a first orientation and a second orientation.

9. The intravascular imaging catheter of claim 8, wherein the imaging device is configured to image at an angle that is substantially normal to the imaging core when the imaging device is in the first orientation.

10. The intravascular imaging catheter of claim 8, wherein the imaging device is configured to image in a distal direction at an angle less than about 90 degrees relative to the imaging core when the imaging device is in the second orientation.

11. The intravascular imaging catheter of claim 1, wherein the imaging device is configured to image in a distal direction at a variable angle.

12. The intravascular imaging catheter of claim 1, wherein the imaging device is mounted upon an inflatable member.

13. The intravascular imaging catheter of claim 12, wherein the imaging device includes a plurality of transducers.

14. The intravascular imaging catheter of claim 12, wherein the inflatable member has a guidewire lumen formed therein.

15. The intravascular imaging catheter of claim 1, wherein the elongate catheter shaft includes a torque-transmitting reinforcing member.

16. An intravascular imaging catheter, comprising:
an elongate catheter shaft having a distal end region and a proximal region;
wherein the proximal region includes a torque-transmitting reinforcing member;
wherein the distal end region includes a side port;
an imaging core disposed within the elongate catheter shaft; and
wherein the imaging core includes one or more ultrasound transducers configured to be slidable within the elongate catheter shaft and through the side port.

17. The intravascular imaging catheter of claim 16, wherein the one or more ultrasound transducers include one or more distally-angled transducers.

18. The intravascular imaging catheter of claim 16, wherein the one or more ultrasound transducers are configured to shift between a first orientation and a second orientation.

19. The intravascular imaging catheter of claim 18, wherein the one or more ultrasound transducers are configured to image at a first angle that is substantially normal to the imaging core when the one or more ultrasound transducers are in the first orientation and wherein the one or more ultrasound transducers are configured to image in a distal direction at a second angle less than about 90 degrees relative to the imaging core when the one or more ultrasound transducers are in the second orientation.

20. A method for imaging a vascular region, the method comprising:
advancing an intravascular imaging catheter through a blood vessel to a position adjacent to an area of interest;
wherein the intravascular imaging catheter comprises:
an elongate catheter shaft having a distal end region and a proximal region,
wherein the distal end region includes a side port,
an imaging core disposed within the elongate catheter shaft, and
wherein the imaging core includes an imaging device configured to be slidable within the elongate catheter shaft and through the side port;
advancing the imaging core so that the imaging device advances through the side port; and
imaging the blood vessel using the imaging device.
